(19) United States
(12) Patent Application Publication
(10) Pub. No.: US 2024/0341707 A1
(43) Pub. Date: Oct. 17, 2024

(54) SYSTEM AND METHOD FOR AUTOMATICALLY DETECTING ORIENTATION AND ANATOMY IN AN IMAGING SYSTEM

(71) Applicant: Medtronic Navigation, Inc., Louisville, CO (US)

(72) Inventors: Christina R. Drake, Harwich, MA (US); Seunghoon Nam, Bedford, MA (US); Andre D. A. Souza, Boylston, MA (US); Patrick A. Helm, Canton, MA (US)

(21) Appl. No.: 18/608,449

(22) Filed: Mar. 18, 2024

Related U.S. Application Data
(60) Provisional application No. 63/458,697, filed on Apr. 12, 2023.
(60) Provisional application No. 63/458,694, filed on Apr. 12, 2023.

(51) Int. Cl. A61B 6/46; G06T 7/00

(52) U.S. Cl. CPC A61B 6/466; A61B 6/463; G06T 7/0012; G06T 2207/30004

(57) ABSTRACT

A method and system for controlling an imaging system includes positioning the imaging system into a first position, acquiring a first image at the first position, determining patient data from the first image, communicating patient data to a user interface, displaying the patient data on a display and acquiring a second image based on the patient data.

### CROSS-REFERENCE TO RELATED APPLICATIONS

This application claims the benefit of and priority to U.S. Provisional Patent Application No. 63/458,697 filed Apr. 12, 2023 and U.S. Provisional Patent Application No. 63/458,694 filed Apr. 12, 2023, and the disclosures of each of the above-identified applications are hereby incorporated by reference in their entirety.

### FIELD

The present disclosure relates to imaging a subject, and particularly to a system to automatically determine the patient orientation to populate a menu system for subsequent images.

### BACKGROUND

This section provides background information related to the present disclosure which is not necessarily prior art.

A subject, such as a human patient, may undergo a procedure. The procedure may include a surgical procedure to correct or augment an anatomy of the subject. The augmentation of the anatomy can include various procedures, such as movement or augmentation of bone, insertion of an implant (i.e., an implantable device), or other appropriate procedures.

When using an imager, various types of data are input by the technician and are used to capture image data. Data such as the imager settings and the patient settings are generally entered prior to capturing the image data. Completing the extensive list or inputting the settings can or is very time consuming and may extend surgical time and/or surgical suite time.

### SUMMARY

This section provides a general summary of the disclosure, and is not a comprehensive disclosure of its full scope or all of its features.

According to various embodiments, a system to acquire image data of a subject may be an imaging system that uses x-rays. The subject may be a living patient (e.g., a human patient). The subject may also be a non-living subject, such as an enclosure, a casing, etc. Generally, the imaging system may acquire image data of an interior of the subject. The imaging system may include a moveable source and/or detector that is moveable relative to the subject. The position and movement of the system is performed automatically to reduce the overall imaging time and provide less exposure of x-rays to the subject.

In various embodiments, the method for controlling an imaging system includes positioning the imaging system into a first position, acquiring a first image at the first position, determining patient data from the first image, communicating patient data to a user interface, displaying the patient data on a display and acquiring a second image based on the patient data.

In another aspect of the disclosure, a system to control an imaging system has a controller configured to execute instructions to acquire a first image at a first position, determine patient data from the first image, communicating patient data to a user interface and displaying the patient data on a display.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is an environmental view of an imaging system in an operating theatre;

FIG. 2 is a detailed schematic view of an imaging system with a source and detector configured to move around a subject, according to various embodiments;

FIG. 3 is a block diagrammatic view of the imaging system.

FIG. 4A is a representation of the order of the vertebrae in a patient.

FIG. 4B is an image of vertebrae illustrating the order of vertebrae.

FIG. 4C is a representation of a patient in a head left supine position.

FIG. 4D is a representation of a patient in a head right supine position.

FIG. 4E is an image of a head left position for the patient in the supine position.

FIG. 4F is a head right position of the patient in the prone position.

FIG. 5A is a representation of a patient data user interface.

FIG. 5B is a representation of an image data user interface.

FIG. 6 is a flowchart of a method for operating the system.

Corresponding reference numerals indicate corresponding parts throughout the several views of the drawings.

### DETAILED DESCRIPTION

Example embodiments will now be described more fully with reference to the accompanying drawings.

A subject may be imaged with an imaging system, as discussed further herein. The subject may be a living subject, such as a human patient. Image data may be acquired of the human patient and may be combined to provide an image of the human patient that is greater than any dimension of any single projection acquired with the imagining system. It is understood, however, that image data may be acquired of a non-living subject, such an inanimate subject including a housing, casing, interior of a super structure, or the like. For example, image data may be acquired of an airframe for various purposes, such as diagnosing issues and/or planning repair work.

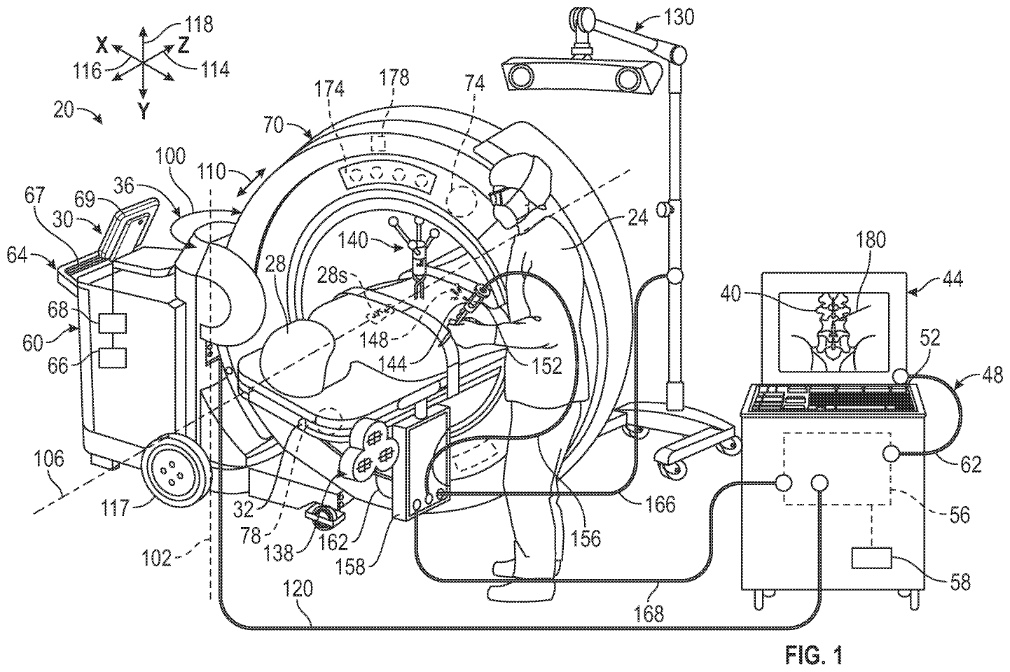

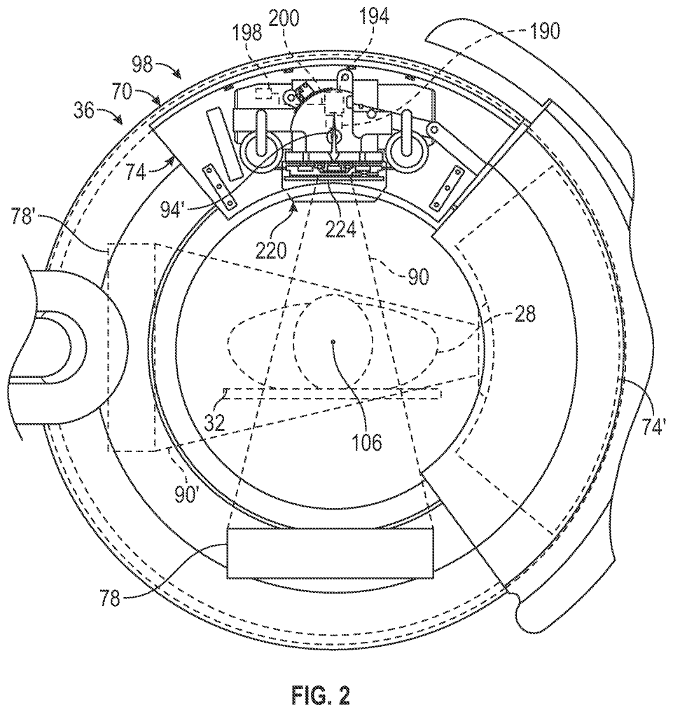

With reference to FIGS. 1 and 2, a schematic view of a procedure room 20 is illustrated. A user 24, such as a surgeon, can perform a procedure on a subject, such as a patient 28. The subject may be placed on a support, such as a table 32 for a selected portion of the procedure. The table 32 may not interfere with image data acquisition with an imaging system 36. In performing the procedure, the user 24 can use the imaging system 36 to acquire image data of the patient 28 to allow a selected system to generate or create images to assist in performing a procedure. Images generated with the image data may be two-dimensional (2D) images, three-dimensional (3D), or appropriate type of images, such as a model (such as a three-dimensional (3D) image), long views, single projections views, etc. can be generated using the image data and displayed as an image 40 on a display device 44. The display device 44 can be part of and/or connected to a processor system 48 that includes a user interface 52, such as a keyboard, mouse, stylus, a touch screen as part of the display device 44 or combinations thereof. A processor 56, can include one or more processors, processor module, and/or microprocessors incorporated with the processing system 48 along with selected types of non-transitory and/or transitory memory 58. A connection 62 can be provided between the processor 56 and the display device 44 for data communication to allow driving the display device 44 to display or illustrate the image 40. The processor 56 may be any appropriate type of processor such as a general-purpose processor that executes instructions included in a program or an application specific processor such as an application specific integrated circuit.

The imaging system 36 can include but is not limited to an O-Arm® imaging system sold by Medtronic Navigation, Inc. having a place of business in Louisville, CO, USA. The imaging system 36, including the O-Arm® imaging system, or other appropriate imaging systems may be in use during a selected procedure, such as the imaging system described in U.S. Patent App. Pubs. 2012/0250822, 2012/0099772, and 2010/0290690, all the above incorporated herein by reference.

The imaging system 36, when, for example, including the O-Arm® imaging system, may include a mobile cart 60 that includes a controller and/or control system 64. The control system 64 may include a processor and/or processor system 68 (similar to the processor 56), a user interface 67 such as a keyboard, a mouse, a touch screen, a memory 58 (e.g., a non-transitory memory) and a display device 69. The memory system 66 may include various instructions that are executed by the processor 68 that acts as a controller to control the imaging system 36, including various portions of the imaging system 36.

The imaging system 36 may include further additional portions, such as an imaging gantry 70 in which is positioned a source unit (also referred to as a source assembly) 74 and a detector unit (also referred to as a detector assembly) 78. In various embodiments, the detector 78 alone and/or together with the source unit may be referred to as an imaging head of the imaging system 36. The gantry 70 is moveably connected to the mobile cart 60. The gantry 70 may be O-shaped or toroid shaped, wherein the gantry 70 is substantially annular and includes walls that form a volume in which the source unit 74 and detector 78 may move. The mobile cart 60 may also be moved. In various embodiments, the gantry 70 and/or the cart 60 may be moved while image data is acquired, including both being moved simultaneously. Also, the imaging system 36 via the mobile cart 60 can be moved from one operating theater to another (e.g., another room). The gantry 70 can move relative to the cart 60, as discussed further herein. This allows the imaging system 36 to be mobile and moveable relative to the subject 28, thus allowing it to be used in multiple locations and with multiple procedures without requiring a capital expenditure or space dedicated to a fixed imaging system.

The processor 68 may be a general-purpose processor or an application specific application processor. The memory system 66 may be a non-transitory memory such as a spinning disk or solid-state non-volatile memory. In various embodiments, the memory system may include instructions to be executed by the processor 68 to perform functions and determine results, as discussed herein. The memory system 66 may be used to store images from the imaging system 36 to allow calculations to be performed thereon. The memory system 66 may be used to store intermediate and final calculations, such as data for identifying body structures, distance for the imaging system to travel, a target position for the imaging system 36.

In various embodiments, the imaging system 36 may include an imaging system that acquires images and/or image data using emitting x-rays and detecting x-rays after interactions and/or attenuations of the x-rays with or by the subject 28. The x-ray imaging may be an imaging modality. It is understood that other imaging modalities are possible, such as other high energy beams, etc.

Thus, in the imaging system 36, the source unit 74 may be an x-ray emitter that can emit x-rays at and/or through the patient 28 to be detected by the detector 78. As is understood by one skilled in the art, the x-rays emitted by the source 74 can be emitted in a cone 90 along a selected main vector 94 and detected by the detector 78, as illustrated in FIG. 2. The source 74 and the detector 78 may also be referred to together as a source/detector unit 98, especially wherein the source 74 is generally diametrically opposed (e.g., 180 degrees) (° apart) from the detector 78 within the gantry 70.

The imaging system 36 may move, as a whole or in part, relative to the subject 28. For example, the source 74 and the detector 78 can move around the patient 28, e.g., a 360° motion, spiral, portion of a circle, etc. The movement of the source/detector unit 98 within the gantry 70 may allow the source 74 to remain generally 180° opposed (such as with a fixed inner gantry or rotor or moving system) to the detector 78. Thus, the detector 78 may be referred to as moving around (e.g., in a circle or spiral) the subject 28 and it is understood that the source 74 remains opposed thereto, unless disclosed otherwise.

Also, the gantry 70 can move isometrically (also referred as “wag”) relative to the subject 28 generally in the direction of arrow 100 around an axis 102, such as through the cart 60, as illustrated in FIG. 1. The gantry 70 can also tilt relative to a longitudinal axis 106 of the patient 28 illustrated by arrows 110. In tilting, a plane of the gantry 70 may tilt or form a non-orthogonal angle with the axis 106 of the subject 28.

The gantry 70 may also move longitudinally in the direction of arrows 114 along the line 106 relative to the subject 28 and/or the cart 60. Also, the cart 60 may move to move the gantry 70. Further, the gantry 70 can move up and down generally in the Y-axis direction of arrows 118 relative to the cart 60 and/or the subject 28, generally transverse to the axis 106 and parallel with the axis 102. The gantry may also be moved in an X direction in the direction of the arrows 116 by moving the wheels 117.

The movement of the imaging system 36, in whole or in part is to allow for positioning of the source/detector unit (SDU) 98 relative to the subject 28. The imaging system 36 can be precisely controlled to move the SDU 98 relative to the subject 28 to generate precise image data of the subject 28. The imaging system 36 can be connected to the processor 56 via a connection 120, which can include a wired or wireless connection or physical media transfer from the imaging system 36 to the processor 56. Thus, image data collected with the imaging system 36 can be transferred to the processing system 56 for navigation, display, reconstruction, etc.

The source 74, as discussed herein, may include one or more sources of x-rays for imaging the subject 28. In various embodiments, the source 74 may include a single source that may be powered by more than one power source to generate and/or emit x-rays at different energy characteristics. Further, more than one x-ray source may be the source 74 that may be powered to emit x-rays with differing energy characteristics at selected times.

According to various embodiments, the imaging system 36 can be used with an un-navigated or navigated procedure. In a navigated procedure, a localizer and/or digitizer, including either or both of an optical localizer 130 and/or an electromagnetic localizer 138 can be used to generate a field and/or receive and/or send a signal within a navigation domain relative to the subject 28. The navigated space or navigational domain relative to the subject 28 can be registered to the image 40. Correlation, as understood in the art, is to allow registration of a navigation space defined within the navigational domain and an image space defined by the image 40. A patient tracker or dynamic reference frame 140 can be connected to the subject 28 to allow for a dynamic registration and maintenance of registration of the subject 28 to the image 40.

The patient tracking device or dynamic registration device 140 and an instrument 144 can then be tracked relative to the subject 28 to allow for a navigated procedure. The instrument 144 can include a tracking device, such as an optical tracking device 148 and/or an electromagnetic tracking device 152 to allow for tracking of the instrument 144 with either or both of the optical localizer 130 or the electromagnetic localizer 138. A navigation/probe interface device 158 may have communications (e.g., wired or wireless) with the instrument 144 (e.g., via a communication line 156), with the electromagnetic localizer 138 (e.g., via a communication line 162), and/or the optical localizer 130 (e.g., via a communication line 166). The interface 158 can also communicate with the processor 56 with a communication line 168 and may communicate information (e.g., signals) regarding the various items connected to the interface 158. It will be understood that any of the communication lines can be wired, wireless, physical media transmission or movement, or any other appropriate communication. Nevertheless, the appropriate communication systems can be provided with the respective localizers to allow for tracking of the instrument 144 relative to the subject 28 to allow for illustration of a tracked location of the instrument 144 relative to the image 40 for performing a procedure.

One skilled in the art will understand that the instrument 144 may be any appropriate instrument, such as a ventricular or vascular stent, spinal implant, neurological stent or stimulator, ablation device, or the like. The instrument 144 can be an interventional instrument or can include or be an implantable device. Tracking the instrument 144 allows for viewing a location (including x,y,z position and orientation) of the instrument 144 relative to the subject 28 with use of the registered image 40 without direct viewing of the instrument 144 within the subject 28.

Further, the imaging system 36, such as the gantry 70, can include an optical tracking device 174 and/or an electromagnetic tracking device 178 to be tracked with the respective optical localizer 130 and/or electromagnetic localizer 138. Accordingly, the imaging system 36 can be tracked relative to the subject 28 as can the instrument 144 to allow for initial registration, automatic registration, or continued registration of the subject 28 relative to the image 40. Registration and navigated procedures are discussed in the above incorporated U.S. Pat. No. 8,238,631, incorporated herein by reference. Upon registration and tracking of the instrument 144, an icon 180 may be displayed relative to, including overlaid on, the image 40. The image 40 may be an appropriate image and may include a 2D image, a 3D image, or any appropriate image as discussed herein.

With continuing reference to FIG. 2, according to various embodiments, the source 74 can include a single assembly that may include a single x-ray tube 190 that can be connected to a switch 194 that can interconnect a first power source 198 via a connection or power line 200. As discussed above, x-rays can be emitted from the x-ray tube 190 generally in the cone shape 90 towards the detector 78 and generally in the direction from the x-ray tube 190 as indicated by arrow, beam arrow, beam or vector 94. The switch 194 can switch power on or off to the tube 190 to emit x-rays of selected characteristics, as is understood by one skilled in the art. The vector 94 may be a central vector or ray within the cone 90 of x-rays. An x-ray beam may be emitted as the cone 90 or other appropriate geometry. The vector 94 may include a selected line or axis relevant for further interaction with the beam, such as with a filter member, as discussed further herein.

The subject 28 can be positioned within the x-ray cone 90 to allow for acquiring image data of the subject 28 based upon the emission of x-rays in the direction of vector 94 towards the detector 78. The x-ray tube 190 may be used to generate two-dimensional (2D) x-ray projections of the subject 28, including selected portions of the subject 28, or any area, region or volume of interest, in light of the x-rays impinging upon or being detected on a 2D or flat panel detector, as the detector 78. The 2D x-ray projections can be reconstructed, as discussed herein, to generate and/or display three-dimensional (3D) volumetric models of the subject 28, selected portion of the subject 28, or any area, region or volume of interest. As discussed herein, the 2D x-ray projections can be image data acquired with the imaging system 36, while the 3D volumetric models can be generated or model image data.

For reconstructing or forming the 3D volumetric image, appropriate techniques include Expectation maximization (EM), Ordered Subsets EM (OS-EM), Simultaneous Algebraic Reconstruction Technique (SART) and Total Variation Minimization (TVM), as generally understood by those skilled in the art. Various reconstruction techniques may also and alternatively include machine learning systems and algebraic techniques. The application to perform a 3D volumetric reconstruction based on the 2D projections allows for efficient and complete volumetric reconstruction. Generally, an algebraic technique can include an iterative process to perform a reconstruction of the subject 28 for display as the image 40. For example, a pure or theoretical image data projection, such as those based on or generated from an atlas or stylized model of a “theoretical” patient, can be iteratively changed until the theoretical projection images match the acquired 2D projection image data of the subject 28. Then, the stylized model can be appropriately altered as the 3D volumetric reconstruction model of the acquired 2D projection image data of the selected subject 28 and can be used in a surgical intervention, such as navigation, diagnosis, or planning. The theoretical model can be associated with theoretical image data to construct the theoretical model. In this way, the model or the image data 40 can be built based upon image data acquired of the subject 28 with the imaging system 36.

With continuing reference to FIG. 2, the source 74 may include various elements or features that may be moved relative to the x-ray tube 190. In various embodiments, for example, a collimator 220 may be positioned relative to the x-ray tube 190 to assist in forming the cone 90 relative to the subject 28. The collimator 220 may include various features such as movable members that may assist in positioning one or more filters within the cone 90 of the x-rays prior to reaching the subject 28. One or more movement systems 224 may be provided to move all and/or various portions of the collimator 220. Further, as discussed further herein, various filters may be used to shape the x-ray beam, such as shaping the cone 90, into a selected shape prior to reaching the subject 28. In various embodiments, as discussed herein, the x-rays may be formed into a thin fan or plane to reach and pass through the subject 28 and be detected by the detector 78.

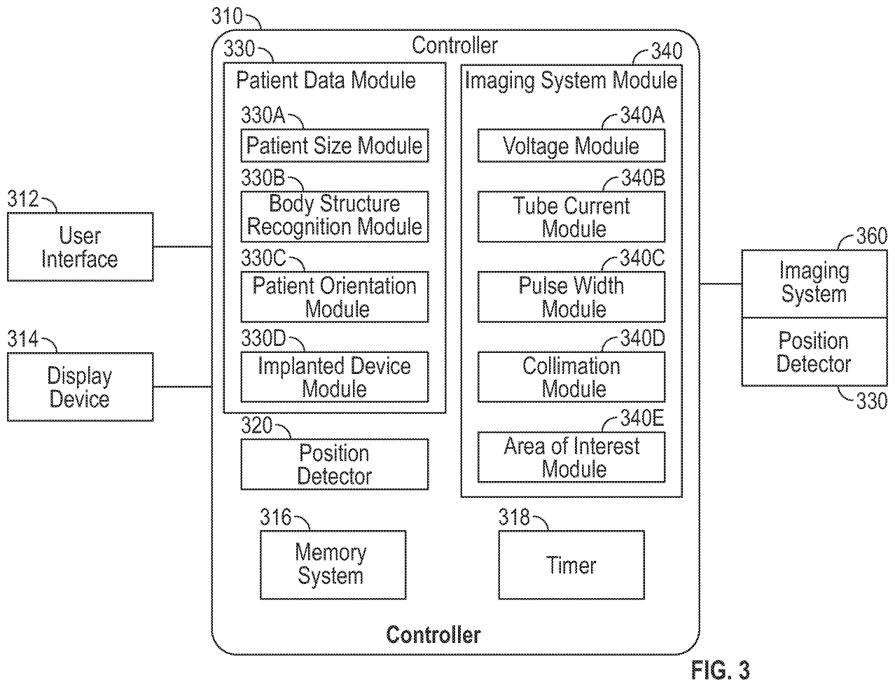

Referring now to FIG. 3, an example of a controller 310 that is programmed to execute instructions is set forth. The controller 310 may be one of or both of the processor 56 and the processor 68. The controller 310 is in communication with a user interface 312. The user interface 312 may be one of or both the user interfaces 52, 67. The user interface may also be used together with a display device 314 that allows selections to be made and data to be entered. The display device 314 may also be referred to as a pendant. The controller 310 is also in communication with the display device 314 which may be one or both of the display devices 44, 69. In summary, the controller 310 may process various signals at the processor 56, the processor 68 or combinations thereof. Likewise, the user interface 312 may provide input to the controller 310 from the user interface 67 or 52. The display device 314 may display various features, images or data at the display device which may be 44 or 69 described above.

The controller 310 includes a memory system 316 that may be one of the memory system 66, the memory system 58 or a combination thereof. The memory system 316 is used to store various data including, but not limited to, the data described above relative to the memory system 66, 58. In addition, the memory system 316 may also be used to store imaging system data such as settings and patient data, both of which will be desired in further detail below.

A timer 318 is used to time various functions including the movement of the imaging system 316.

In the following example, the controller 310 is used to position the imaging system 36 having an O-arm. Of course, other variations of the imaging system 36 may be used. The imaging system 36 may have a position detector 320 associated therewith. The position detector 320 is used for determining the relative position of the O-arm or movable structure of the imaging system 36. A relative position relative to a subject 28 may be obtained. The position detector 320 may include encoders that are used to determine the amount of movement from a predetermined or an initial position. The position of various portions relative to others may also be determined with any one or more appropriate position determination systems. As discussed herein, the position of one or more portions are used to assist in determining an appropriate setup (e.g., initial) of the imaging system.

The controller 310 may also include a patient data module 330. The patient data module 330 may be used to calculate or determine various patient data based upon an image from the imaging system 336. In this example, a two-dimensional image may be used to determine various patient data.

The patient data module 330 includes a patient size module 330A. The patient size module 330A may include and/or be used to determine the size and/or geometry of the patient as a whole. For example, the patient size module 330A may be used to determine a width of the patient from side to side, such as a should or abdomen width. The patient size module 330A may further determine a thickness of the patient anterior to posterior. The patient size module 330A measures the sizes of the patient from the image from the imaging system.

A body structure recognition module 330B may also be incorporated into the patient data module 330. The body structure recognition module 330B may recognize various structures within the body that are within the image from the imaging system 36. Examples of different types of body structures will be provided below. For example, vertebrae and the orientation and order of the vertebrae may allow various data regarding the position of the patient to be determined. That is, in 330C, a patient orientation module uses the body structure that is recognized within the body structure recognition module to determine the patient orientation. Examples of patient orientation are prone or supine. The patient orientation module 330C also recognizes head first or feet first relative to an O-arm. An existing device or implanted device module 330D may provide a location of an existing device or devices within the body. For example, artificial knees, hips, shoulders, spinal implants and pacemakers are some of the types of existing devices that may be identified and located relative to the patient. The implanted device module 330D may provide a coordinate for an existing device without identifying the device. However, based upon the image from the imaging system 36, the type of device may also be recognized. Types of recognition may include neural networks and machine learning that form a trained classifier for determining the existing types of devices within a body. Other types of recognition including using atlas data, segmentation, tables and databases of implantable shapes and geometries of implantable devices may be used.

The controller 310 may also include an imaging system module 340. The imaging system module 340 provides data for the settings of the particular imager and the X-ray tube therein. That is, different imaginers require different types of settings and therefore the exact types of data may vary. In this example, a voltage module 340A may determine the amount of voltage required by the imaging system. Data from the patient data module 330 may be used in this calculation. How large the patient is in terms of AP thickness, the type of body structure to be imaged (tissue, hard bone, soft bone) and width allow the voltage module 340A to determine an amount of voltage to be used at the imaging system. For example, the values for X-ray tube amperage (e.g., milliamps (mA)) and/or of voltage (e.g., kilovolts or kilovolt peak (kVp)), or X-ray beam filtration may be determined. The imaging system module 340 may also include a tube current module 340B. The tube current module 340B may provide a tube current so an adequate image is obtained. The tube current module 340B depends upon various body structures and the size of the patient, the type of body structure to be imaged and can be calculated.

A pulse width module 340C is used to determine the pulse width of the beam generated from the imaging system. Again, various patient data, such as the size of the patient (width and thickness), the body structure that will be altered in the procedure and implantable devices may also have an effect on the pulse width.

A collimation module 340D is used to determine the type of collimation for the imaging system. Again, the collimation module 340D may change the collimation of the imaging system 36 based upon various patient data including the size, the body structure to be modified, the patient orientation and any existing devices that are located within the patient. Collimation change the shape of the beam used for imaging. Collimation can be used to remove highly attenuating (Metallic structure) or lightly attenuating (Air) objects so that the technique factor (KVP, pulse width, mA, beam filtration) can be optimized to visualize anatomy.

The area of interest module 340E is also disposed within the imaging system module. The area of interest module 340E determines the area of interest to be scanned based upon the body structure, the patient size and orientation determined at the patient data module 330. The area of interest module 340E thus provides the desired position of the detector and the emitter of the imaging system to obtain the desired image of the body structure of interest.

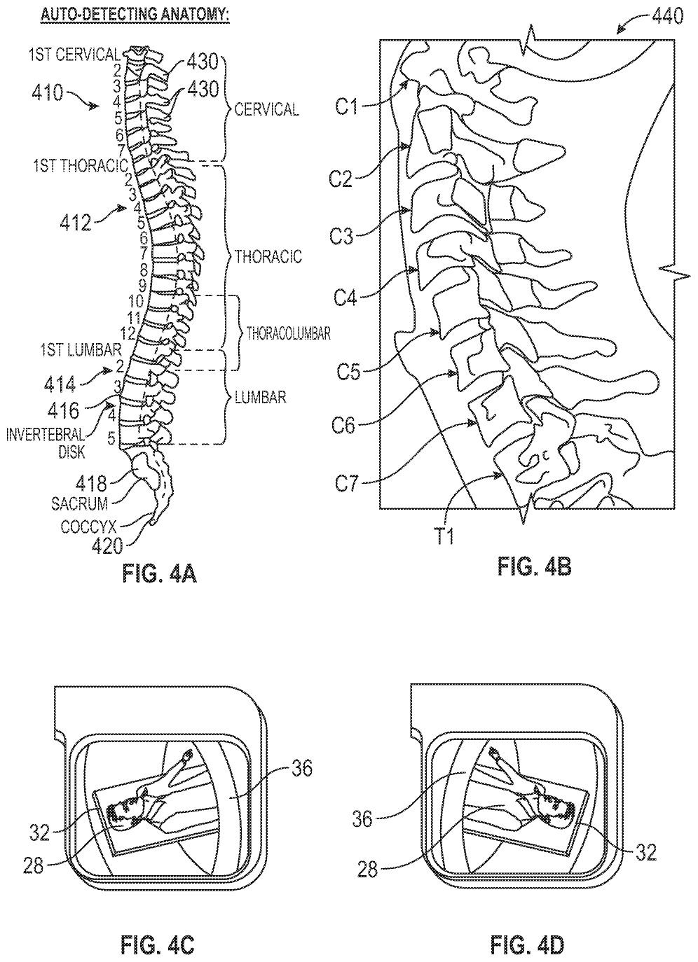

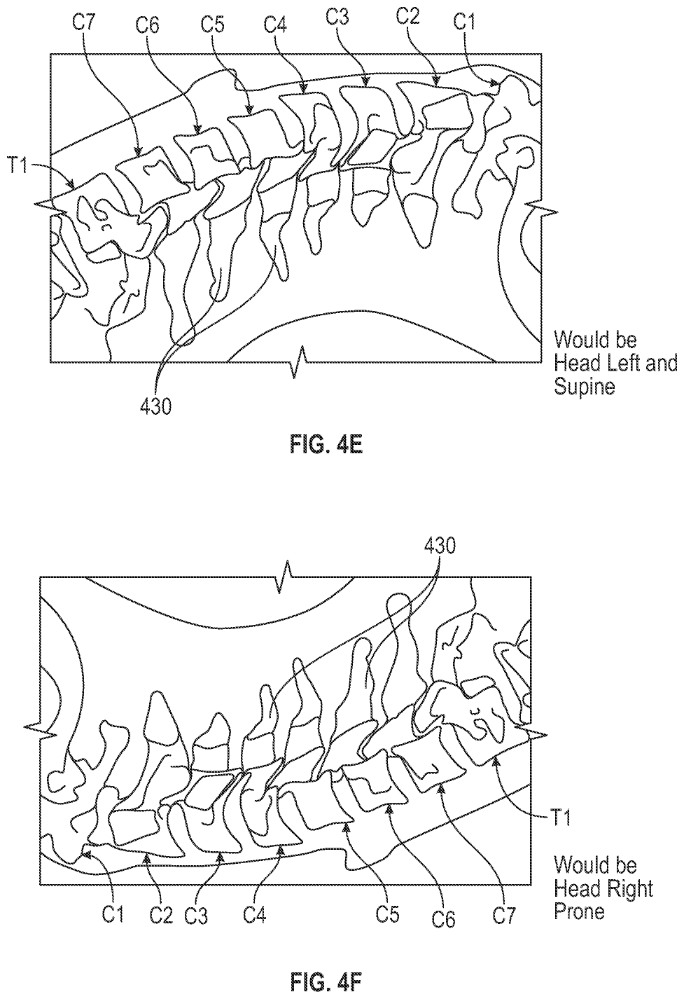

Referring now to FIG. 4A, an example of a body structure is illustrated. The body structure may include but is not limited to vertebrae, an end plate, corners of an end plate, a full vertebrate, a partial vertebrate, a skull, a limb, or an organ. The vertebrae of a patient are illustrated in FIG. 4A. The spinal column body structure includes cervical vertebrae 410, thoracic vertebrae 412, lumbar vertebrae 414, and intervertebral disk 416, a sacrum 418 and the coccyx 420. Because the vertebrae have a particular order and the order can be recognized by the body structure recognition module 330B, the orientation of the patient may be determined based on the known position of the imager. The spinous process 430 extend from each of the vertebrae and allows the orientation in terms of supine and prone positions to be recognized by the controller 310.

Referring now to FIG. 4B, a representation of vertebrae C1-T1 is shown. An image 440 of this type would recognize the body structure in the body structure recognition module as a chest category.

Referring now to FIGS. 4C and 4D, a representative patient 28 being oriented relative to the imaging system 36 is set forth. In this example, FIG. 4C illustrates the patient in a head left supine position. FIG. 4D illustrates a head right supine position. The orientations illustrated in FIGS. 4C and 4D may be identified by labeling the vertebrae, the order of the vertebrae and the spinous process as described above and the position of the image when the image is taken.

Referring now to FIG. 4E, a head left position or orientation of the patient 28 is illustrated based upon the position of the spinous process 430 and the order of the label vertebrae C1-T1.

Referring now to FIG. 4F, the patient is illustrated in a head right position based upon the order of the vertebrae. Likewise, by knowing the position of the rotor when the imaging system 36 obtains the two-dimensional image, FIGS. 4E and 4F illustrate a supine and prone position, respectively. That is, in FIG. 4F, the spinous process 430 is in an up position. If the rotor is in LAT position, the patient is prone relative to the detector. If the rotor is in an AP position, the patient is lateral relative to the detector.

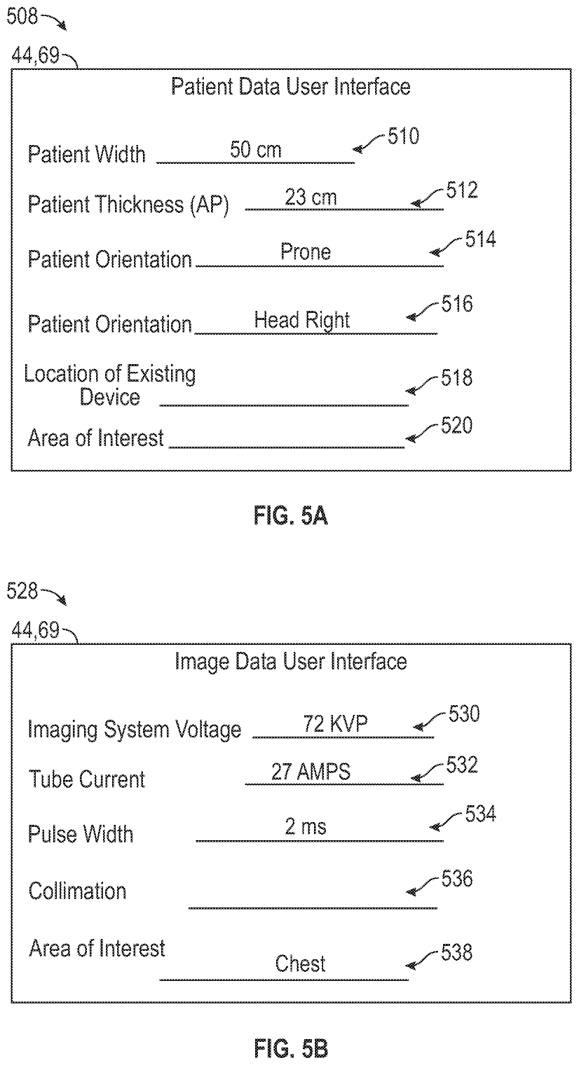

Referring now to FIG. 5A, a patient data user interface that displays patient data is displayed on one of the screen displays 44, 69 is illustrated. As mentioned above, various types of patient data may be provided and displayed beyond that set forth in FIG. 5A.

In this example, the user interface 508 has a patient width 510, a patient thickness 512, a patient orientation such as prone or supine 514 and head right or head left 516 and a location of existing devices 518 such as prior implants. The area of interest 520 may also be a user interface selection.

Referring now to FIG. 5B, a user interface 528 includes an image data user interface 528 that is a three-dimensional image user interface that includes data and/or settings used for taking three-dimensional images. As mentioned above, a two-dimensional image may be obtained and the patient data and ultimately image data may be provided based upon the patient data. In this example, the imaging system power 530, the tube current 532, the pulse width 534, the collimation 536 and the area of interest 538 are determined automatically based on the above. As mentioned above, the area of interest includes an area of the body that will be imaged. The area of interest allows the detector and the emitter of the imaging system to be aligned properly.

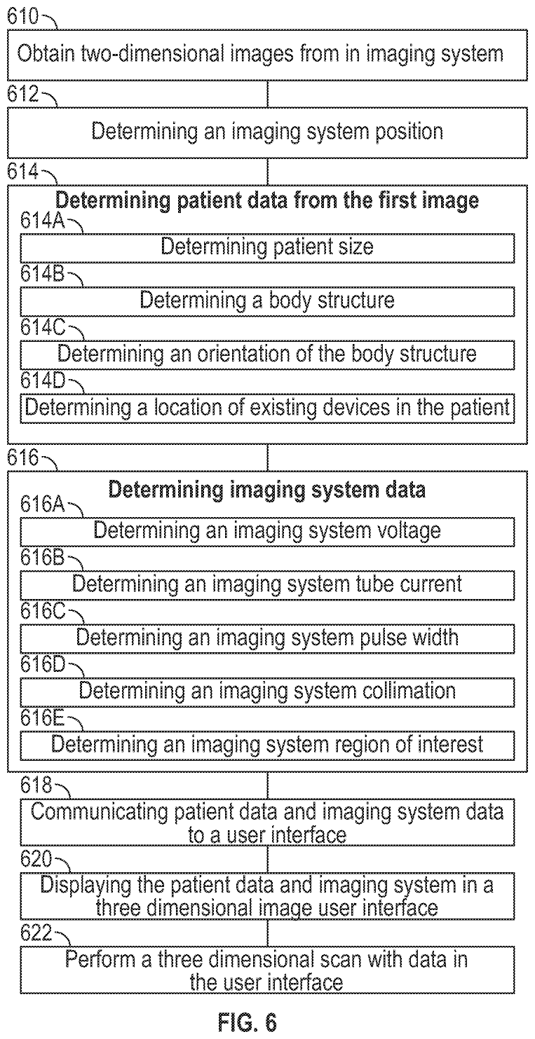

Referring now to FIG. 6, a method for operating the imaging system 36 is set forth. In this example, a two-dimensional image is obtained from the imaging system in block 610. In block 612, the imaging system position is also obtained from the imaging system. That is, the position of the detector and/or the emitter are provided to the controller.

Block 614 obtains patient data from the two-dimensional image of block 610. Block 614 is broken down into a plurality of sub blocks. In block 614A, the patient size is obtained. The patient size may include the width of the patient and the AP thickness of the patient. The patient size may be determined by evaluating measurements determined from the image data in block 610. In the block 614B, a body structure of the patient may be determined and its position. That is, the position of the imaging system is relative to a particular one or more body structures of the patient such as by evaluating the image data from block 610 and the determined position of the imaging system from block 612 . . . . Examples are illustrated in FIG. 4A. When the body structure is identified in block 614B, the orientation of the body may be obtained in block 614C. That is, the position of the skull may be identified in the image from block 610 and if the position of the skull is known or the position of the vertebras and the order of the vertebras, the patent orientation may be known. Likewise, when the spinous process is identified, the prone or supine position of the patient may be determined. In block 614D, a location of existing devices may be obtained. That is, the location of existing devices including, but not limited to, implants, plates, replacement joints and pacemakers may be determined in the image data from block 610. As the image obtained in block 610 may be used to identify various features and portions therein and the position of the imaging system is determined in block 612, the relative position of the imaging system to the patient may be determined and the patient data in block 614 may be determined.

Ultimately, the patient data in block 614 is used to populate the patient data user interface. That is, the various patient data determined using the initial or test images in block 610 may be used to populate various fields of the patient data user interface as noted above. The user may then review the completed fields for various purposes, such as verification thereof.

The patient data may also be used to determine the various data of the imaging system in block 616 to be used to take a next (e.g., second) or diagnostic image at the controller, such as a three-dimensional image and/or a plurality of 2D images that may be reconstructed into a 3D image. Block 616 has various sub blocks that determine the settings for the imaging system. In the various sub blocks parameters of the imaging system may be determined or recalled based on the patient data from block 614.

In block 616A, an imaging system voltage may be determined. That is, the amount of voltage based upon the patient data may be determined and obtained. For example, larger patients may require more voltage to take an adequate image with the proper contrast. The voltage amount may be recalled and/or determined based on a selected determined patient size.

In block 616B, an imaging system tube current may be determined by the controller. The tube current may use the patient size data and the orientation in a similar manner to that described above relative to the system power. The power of the system uses both the voltage and the current. In block 616C, the pulse width of the beam may be determined. Depending upon the contrast desired, the imaging system pulse width may vary.

In block 616D, an imaging system collimation may be provided, particularly to collimate a beam, which may include altering a beam, such as a beam of x-rays. Collimation of the beam can be used to remove highly attenuating (metallic structure) or lightly attenuating (air) objects so that the technique factor (KVP, pulse width, mA, beam filtration) can be optimized to visualize anatomy. In other words, the collimation may be done, such as with a collimator, to shape and/or direct the beam. In so doing, the beam may be directed to not engage or not pass through highly attenuating items or lightly attenuating items to optimize or enhance image data acquired through the selected subject, also referred to as field of interest.

In block 616E, an imaging system region of interest may be identified. The region of interest may be identified from the patient size data and the body structure described above. The imaging system region of interest allows the detector and emitter of the imaging system to be oriented in a selected position (e.g., direction for emitting x-rays) relative to the patient to acquire image data for the purposes. For example, image data to reconstruct a three dimensional image and/or acquire three dimensional image data. The imaging system 36, including only portions thereof, may be moved as discussed above.

In block 618, the patient data and the imaging system data are communicated to a user interface. As illustrated in FIGS. 5A and 5B, two different user interfaces may be generated. However, the user interfaces may also be combined or in a list that may be scrolled through in order to see the various data. The user interface may also include a menu and sub menu display type and/or a graphical display that may include graphics of the imaging system 36 and/or the patient 28. The patient data and the imaging system data may be used by the imaging system to acquire a selected or required subsequent image data. The patient data and the imaging system data, therefore, may be determined only automatically and/or with little manual input as an input for setting up the imaging system to acquire subsequent image data (e.g., diagnostic image data) as discussed herein.

In block 620, the patient data and the imaging system data may be displayed in a user interface for obtaining a subsequent image data acquisition, which may be a three-dimensional image. As mentioned above, examples of the three-dimensional image user interfaces are set forth in FIGS. 5A and 5B. The user interface may be displayed on a screen display of the imager.

In block 622, a diagnostic image scan, which may be a three-dimensional scan, is performed with the data as included in the user interface. The imaging system data, as well as the patient data, may be used to obtain the three-dimensional image in block 622. The imaging system data and/or the patient data allows the imaging system 36 to be setup and operated to generate the image data that is selected of the patient 28. This may include the area of interest and of a selected quality and/or contrast for analysis and/or diagnoses. As discussed above, the imaging system data and the patient data, however, may be substantially automatically obtained without manual intervention. This may increase consistency and/or reduce operating room time and/or radiation exposure.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors or processor modules, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A method of controlling an imaging system comprising:
positioning the imaging system into a first position;
acquiring a first image at the first position;
determining patient data from the first image;
communicating patient data to a user interface;
displaying the patient data on a display; and
inputting the patient data configured for acquiring second image.

2. The method of claim 1 wherein determining patient data comprises determining a patient width and patient AP thickness from the first image.

3. The method of claim 1 wherein determining patient data comprises determining a first body structure from the first image.

4. The method of claim 3 wherein determining the first body structure comprises determining vertebrae.

5. The method of claim 3 wherein determining the first body structure comprises determining at least one of an end plate, corners of an end plate, a full vertebrate, a partial vertebrate, a skull, a limb, or an organ.

6. The method of claim 3 further comprising determining an orientation of the first body structure based on the first position and the first image.

7. The method of claim 6 wherein determining the orientation comprises determining an orientation of vertebrae.

8. The method of claim 6 wherein determining the orientation comprises determining a prone position or a supine position.

9. The method of claim 6 wherein determining the orientation comprises determining a direction of a spinous process.

10. The method of claim 6 wherein determining the orientation comprises determining a lateral left orientation or lateral right orientation.

11. The method of claim 1 wherein acquiring the first image comprises acquiring the first image comprising a first two-dimensional image.

12. The method of claim 1 wherein the user interface is a three-dimensional image user interface and further comprising acquiring a three-dimensional image using the data.

13. The method of claim 1 further comprising determining imaging system data based on the patient data and the first image, communicating the imaging system data to the user interface and displaying patient data and imaging system data on the display.

14. The method of claim 13 wherein determining imaging system data comprises determining imaging system voltage.

15. The method of claim 13 wherein determining imaging system data comprises determining imaging system tube current.

16. The method of claim 13 wherein determining imaging system data comprises determining imaging system pulse width.

17. The method of claim 13 wherein determining imaging system data comprises determining imaging system collimation.

18. The method of claim 13 wherein determining imaging system data comprises determining imaging system region of interest.

19. The method of claim 13 wherein determining imaging system data comprises determining at least three of imaging system power, imaging system tube current, imaging system pulse width, imaging system collimation, and imaging system region of interest.

20. A system to control an imaging system, the system comprising:
a controller configured to execute instructions to,
acquire a first image at a first position;
determine patient data from the first image;
communicating patient data to a user interface;
displaying the patient data on a display; and
acquire a second image based on the patient data.

21. The system of claim 20 wherein the patient data comprises a patient width and patient thickness from the first image.

22. The system of claim 20 wherein the patient data comprises a first body structure from the first image.

23. The system of claim 22 wherein the first body structure comprises vertebrae.

24. The system of claim 22 wherein the first body structure comprises at least one of an end plate, corners of an end plate, a full vertebrate, a partial vertebrate, a skull, a limb, or an organ.

25. The system of claim 22 wherein the controller is configured to execute instructions to determine an orientation of the first body structure based on the first position and the first image.

26. The system of claim 25 wherein the orientation comprises an orientation of vertebrae.

27. The system of claim 25 wherein the orientation comprises a prone position or a supine position.

28. The system of claim 25 wherein the orientation comprises a direction of a spinous process.

29. The system of claim 25 wherein the orientation comprises a lateral left orientation or lateral right orientation.

30. The system of claim 20 wherein the first image comprises a first two-dimensional image.

31. The system of claim 20 wherein the user interface is a three-dimensional image user interface and wherein the controller is configured to execute instructions to acquire a three-dimensional image using the data.

32. The system of claim 20 wherein the controller is configured to execute instructions to determine imaging system data based on the patient data and the first image, communicate the imaging system data to the user interface and display patient data and imaging system data on the display.

33. The system of claim 32 wherein the imaging system data comprises imaging system power.

34. The system of claim 32 wherein the imaging system data comprises imaging system tube current.

35. The system of claim 32 wherein the imaging system data comprises imaging system pulse width.

36. The system of claim 32 wherein the imaging system data comprises imaging system collimation.

37. The system of claim 32 wherein the imaging system data comprises imaging system region of interest.

38. The system of claim 32 wherein the imaging system data comprises at least three of imaging system power, imaging system tube current, imaging system pulse width, imaging system collimation, and imaging system region of interest.
